(19) United States
(12) Patent Application Publication
(10) Pub. No.: US 2009/0047362 A1
(43) Pub. Date: Feb. 19, 2009

(54) Method for in-vitro stabilization of heavy metals

(76) Inventor: Keith Edward Forrester, Meredith, NH (US)

Correspondence Address: Keith E. Forrester, 78 Tracy Way, Meredith, NH, 03253, US

(21) Appl. No.: 12/069,651

(22) Filed: Feb. 12, 2008

Related U.S. Application Data
(60) Provisional application No. 60/964,582, filed on Aug. 13, 2007.

(51) Int. Cl. A61K 33/42; A61K 33/14; A61K 33/26

(52) U.S. Cl. 424/602; 424/646; 424/678; 424/679; 424/680

(57) ABSTRACT

This invention provides a method for reducing the level of 1.0 Normal HCL soluble heavy metals within the human stomach acid environment by oral ingestion of stabilizing agents such that the heavy metal solubility is reduced and subsequent heavy metal transfer to the blood stream is reduced to acceptable levels or eliminated.

### BACKGROUND OF THE INVENTION

The human health hazard of ingestion and subsequent digestion and blood uptake of Lead (Pb), Cadmium (Cd), Chromium (Cr), Arsenic (As) and other heavy metals from natural sources, man-made materials and commercial and industrial wastes, has been the basis for extensive regulatory control and toxicological study for many decades. Recent studies by the Center for Disease Control (CDC) suggest that there are no safe levels of Pb in the blood of infants, as the soluble lead in the blood stream has a direct negative impact on brain development and reduces IQ, as well as having other negative impacts on renal and liver development.

There exist numerous exposure routes which heavy metals can come in contact with humans, such as ingestion of water soluble and water insoluble lead and arsenic in drinking water supplies, ingestion of heavy metals in plant cellulose from pesticide and herbicide usage in crop production, dermal adsorption of surficial soils contaminated with heavy metal wastes, dermal adsorption and ingestion of surficial soils contaminated with lead paint, dermal adsorption, inhalation and ingestion of surficial soils and surface rainwater runoff and leachates contaminates with organic tetraethyl lead from automobile exhaust, adsorption of lead stabilizer in plastic material surfaces, adsorption of color inks and agents in papers and magazines, adsorption of lead bearing PVC stabilizer on copper wire, and inhalation and ingestion of particulates not captured from APC devices such as cyclones, electrostatic precipitators and bag-house filters at industrial and domestic waste incinerators, smelters, steel mills, foundries, casting plants, furnaces, and combustors.

Currently throughout most of the world, high leachable content heavy metal bearing wastes, i.e., regulated Hazardous Wastes, produced from industrial and commercial sources are regulated and managed in a safe manner to limit exposure to humans and the environment. Even with this regulation of high content industrial and commercial wastes, there remain numerous routes of exposure of heavy metal bearing materials and wastes to humans that are not regulated as well as residual heavy metals in our environment from past unregulated sources, and thus there exists unsafe heavy metal exposure to humans which can remain in the workplace, home, and common areas such as playgrounds, lakes, rivers, ponds, roadsides, and perimeter of buildings and structures. One of the most studied and common routes of exposures to children are lead bearing soils and leaded paints, and exposure to lead and arsenic from drinking water sources, both natural and man-made minerals. There also exists heavy metal ingestion exposure from fish, certain dairy products, crops such as rice grown in heavy metal bearing soils, certain painted toys, and even newspapers and magazines, where certain heavy metals are used for color enhancers.

The exposure to heavy metals in infants and children, with the exception of certain groundwater and surface water sources and foods, is most commonly a solid heavy metal source such a paint chip or paint particle, which is ingested by hand-to-mouth contact randomly of with food, and subsequently digested in the stomach by a 1.0 normal hydrochloric stomach acid (produced by our internal digestive acid-producing glands), converting from a solid phase to the acid soluble form of heavy metal, before transfer through the stomach and intestine walls to the bloodstream. Once the acid soluble heavy metal is in the bloodstream, the soluble heavy metals now have the ability to travel to various organs such as the liver and brain, where cellular damage begins.

Given that empirical testing of heavy metal exposure to infants and children is illegal and unethical, and animal studies are unethical and expensive, a repeatable bench scale test was developed by the inventor in order to simulate the human digestive system and to study solid and soluble-phase in-vitro heavy metal stabilization kinetics. The Forrester In-Vitro Leach Test (FILT) uses a Hydrochloric (HCL) acid batch leach test where 50 grams of subject heavy metal bearing material are tumbled in a 1000 ml tumbler with 500 grams of 1.0 Normal HCL solution for a period of 48 hours. The heavy metal concentration is then analyzed by Inductively-Coupled Plasma (ICP) after filtration of a 100 ml aliquot from the tumbler through a 0.75 micron glass bead filter and digestion. Stabilized heavy metal bearing solids not digested in HCL would be expected to pass the human stomach and intestines and thus discharge in fecal matter as inert mass.

The present invention provides a reactive post-exposure in-vitro iron, chloride, and phosphate combination based non-toxic, non-carcinogenic, ingestible heavy metal stabilization method for reducing the HCL solubility of the solid phase and soluble heavy metal sources introduced into the human stomach. The method reduces or eliminates the ability of ingested heavy metals to digest in the stomach and subsequently transfer from the stomach into the bloodstream. By converting the previously soluble heavy metal molecule(s) into HCL insoluble mineral(s) would allow the newly formed heavy metal mineral(s) to pass the stomach and intestine and be discharged in fecal matter. By providing iron, chloride, phosphates, and combinations thereof, in water insoluble, water soluble or water semi-soluble sources in the stomach during periods of exposure to the heavy metal bearing environment, the stomach will act as a Reactive Continuous Flow Stirred Tank Reactor (CFSTR), thus allowing certain heavy metals to form into HCL-insoluble mineral form, and thus pass the stomach as an inert solid and avoid transfer into the hosts bloodstream.

The heavy metal groups capable of stabilization in the stomach by exposure to certain concentrations of iron, chloride, monocalcium, dicalcium and tricalcium phosphates, include mineral groups As, Ag, Cd, Cr, Pb, Hg, Se, Sb, Cu, Ni, Zn, and combinations thereof.

Unlike the present invention, prior art has taught stabilization of heavy metals prior to human exposure and adsorption, inhalation and/or ingestion, to reduce the heavy metal leachability under water, DI water, dilute acetic acid, dilute citric acid, dilute sulfuric acid and dilute nitric acid, to meet certain regulatory limits under hazardous waste or groundwater regulations. All of these methods did not teach use of a stabilizer suitable for human ingestion and allowed by the Food and Drug Administration, suitable for stabilization in-vitro, and at a low cost to allow affordable extended ingestion of the stabilizer by children and adults routinely exposed to heavy metals in the workplace or living areas. Given the extensive environmental, community and living space sources of heavy metal exposure to infants, children and adults, it is deemed necessary that these populations can only be provided ample protection from heavy metals by use of a reactive means, which remains with the exposed party regardless of heavy metal source exposures and duration of exposures. Proactive heavy metal exposure reduction or stabilization is also deemed too expensive and limited to point sources where remedial budgets exist.

O'Hara (U.S. Pat. No. 4,737,356) and Forrester (U.S. Pat. Nos. 5,245,114 and 5,430,233) teach the need to add water soluble phosphates to incinerator ash, auto shredder and wire insulation wastes, and incinerator bottom ash, which are at least 5 gm/100 ml water solubility, with the preferred embodiment being 100% water soluble phosphoric acid.

U.S. Pat. No. 5,202,033 describes an in-situ method for decreasing Pb TCLP leaching from solid waste using a combination of solid waste additives and additional pH controlling agents from the source of phosphate, carbonate, and sulfates.

U.S. Pat. No. 5,037,479 discloses a method for treating highly hazardous waste containing unacceptable levels of TCLP Pb such as lead by mixing the solid waste with a buffering agent selected from the group consisting of magnesium oxide, magnesium hydroxide, reactive calcium carbonates and reactive magnesium carbonates with an additional agent which is either an acid or salt containing an anion from the group consisting of Triple Superphosphate (TSP), ammonium phosphate, diammonium phosphate, phosphoric acid, boric acid and metallic iron.

### SUMMARY OF THE INVENTION

The present invention discloses a reactive in-vitro heavy metal stabilization method through contact of the heavy metals in the stomach with non-toxic and ingestible forms of Iron (Fe), Chloride (Cl), Monocalcium phosphate (MCP), Dicalcium phosphate (DCP), Tricalcium phosphate (TCP), phosphates and combinations thereof. The iron, chloride, and phosphate would be ingested at a rate and in a form that would provide the subject with a tolerance to certain ingestion, inhalation, and adsorption levels of heavy metals.

The preferred phosphate component embodiment would be DiCalcium Phosphate, which is commonly used as a binder for medicines, meat aging stabilizer, and has existing FDA approval as well as extensive human ingestion exposure study and determined as an inert digestive agent. The Fe, Cl, MCP, DCP, TCP, phosphate and combinations could also be modified by coatings to permit a time-release within the stomach and intestine thus providing for a more effective spanning of the exposure period of heavy metal metals to the stabilizing agents during the entire digestion period.

### DETAILED DESCRIPTION

There exist numerous exposure routes which heavy metals can come in contact with humans, such as ingestion of water soluble and water insoluble lead and arsenic in drinking water supplies, ingestion of heavy metals in plant cellulose from pesticide and herbicide usage in crop production, dermal adsorption of surficial soils contaminated with heavy metal wastes, dermal adsorption and ingestion of surficial soils contaminated with lead paint, dermal adsorption, inhalation and ingestion of surficial soils and surface rainwater runoff and leachates contaminates with organic tetraethyl lead from automobile exhaust, adsorption of lead stabilizer in plastic material surfaces, adsorption of color inks and agents in papers and magazines, adsorption of lead bearing PVC stabilizer on copper wire, and inhalation and ingestion of particulates not captured from APC devices such as cyclones, electrostatic precipitators and bag-house filters at industrial and domestic waste incinerators, smelters, steel mills, foundries, casting plants, furnaces, and combustors.

Currently throughout most of the world, high leachable content heavy metal bearing wastes, i.e., regulated Hazardous Wastes, produced from industrial and commercial sources are regulated and managed in a safe manner to limit exposure to humans and the environment. Even with this regulation of high content industrial and commercial wastes, there remain numerous routes of exposure of heavy metal bearing materials and wastes to humans that are not regulated as well as residual heavy metals in our environment from past unregulated sources, and thus there exists unsafe heavy metal exposure to humans which can remain in the workplace, home, and common areas such as playgrounds, lakes, rivers, ponds, roadsides, and perimeter of buildings and structures. One of the most studied and common routes of exposures to children are lead bearing soils and leaded paints, and exposure to lead and arsenic from drinking water sources, both natural and man-made minerals. There also exists heavy metal ingestion exposure from fish, certain dairy products, crops such as rice grown in heavy metal bearing soils, certain painted toys, and even newspapers and magazines, where certain heavy metals are used for color enhancers.

The exposure to heavy metals, with the exception of certain groundwater and surface water sources and foods, is most commonly a solid heavy metal source such a paint chip or paint particle, which is ingested by hand-to-mouth contact randomly of with food, and subsequently digested in the stomach by a 1.0 normal hydrochloric stomach acid (produced by our internal digestive acid-producing glands), converting from a solid phase to the acid soluble form of heavy metal, before transfer through the stomach and intestine walls to the bloodstream. Once the acid soluble heavy metal is in the bloodstream, the soluble heavy metals now have the ability to travel to various organs such as the liver and brain, where cellular damage begins.

Given that empirical testing of heavy metal exposure to infants and children is illegal and unethical, and animal studies are unethical and expensive, a repeatable bench scale test was developed by the inventor in order to simulate the human digestive system and to study solid and soluble-phase in-vitro heavy metal stabilization kinetics. The Forrester In-Vitro Leach Test (FILT) uses a Hydrochloric (HCL) acid batch leach test where 50 grams of subject heavy metal bearing material are tumbled in a 1000 ml tumbler with 500 grams of 1.0 Normal HCL solution for a period of 48 hours. The heavy metal concentration is then analyzed by Inductively-Coupled Plasma (ICP) after filtration of a 100 ml aliquot from the tumbler through a 0.75 micron glass bead filter and digestion. Stabilized heavy metal bearing solids not digested in HCL would be expected to pass the human stomach and intestines and thus discharge in fecal matter as inert mass.

An object of the invention is to reduce soluble heavy metals in the bloodstream by providing humans an oral stabilizer which will establish a reactive in-vitro mineral iron, chloride, and/or phosphate singular or combination environment which will have the impact of reducing the solubility of the solid phase and soluble heavy metal sources introduced into the stomach, and thus reducing or eliminating the ability of the heavy metal to transfer from the stomach to the bloodstream. The newly established phosphate-based apatite heavy metal, now being insoluble in hydrochloric stomach acid, would pass the stomach and intestine and be discharged safely in fecal matter. By providing iron, chloride, and calcium phosphates, in singular form and combinations in insoluble, soluble or semi-soluble sources in the stomach during periods of exposure to the heavy metal bearing environment, the stomach will act as a Continuous Flow Stirred Tank Reactor (CFSTR), thus allowing certain heavy metals to form into HCL-insoluble mineral form, and thus pass the stomach as an inert solid and avoid transfer into the hosts bloodstream.

The heavy metal groups capable of stabilization in the stomach by exposure to iron, chloride, monocalcium phosphate, dicalcium phosphate, tricalcium phosphate, phosphates, and combinations would include mineral groups As, Ag, Cd, Cr, Pb, Hg, Se, Sb, Cu, Ni, Zn, and combinations thereof. The most likely produced mineral for Pb control would be a calcium phosphate apatite, where calcium would be exchanged in the apatite molecule by Pb. The calcium phosphate based apatite has also been shown to exchange calcium for As, Cd, Cr, and Zn, under hazardous waste leaching tests reported by Forrester. The introduction of combinations or iron and chloride will enhance the formation of chloropyromorphite and corkite, and possibly certain ferric arsenic complexes insoluble at the low pH environment of the human stomach.

Unlike the present invention, prior art has taught stabilization of heavy metals prior to human ingestion, to reduce the heavy metal leachability under water, DI water, dilute acetic acid, dilute citric acid, dilute sulfuric acid and dilute nitric acid, to meet certain regulatory limits under hazardous waste or groundwater regulations. All of these methods did not teach use of a oral stabilizer suitable for human ingestion and allowed by the Food and Drug Administration, suitable for stabilization in-vitro, and at a low cost to allow affordable extended ingestion of the stabilizer by children and adults routinely exposed to heavy metals in the workplace, living areas, and/or foods.

Examples of suitable oral stabilizing agents include, but are not limited to, iron powder, ferric sulfate, ferric chloride, sodium chloride, calcium chloride, magnesium oxide, phosphate rock, pulverized phosphate rock, calcium orthophosphates, monocalcium phosphate, dicalcium phosphate, tricalcium phosphate, hypophosphoric acid, metaphosphoric acid, hexametaphosphate, tertrapotassium polyphosphate, polyphosphates, phosphoric acid, technical grade phosphoric acid, wet process amber and green phosphoric acid, trisodium phosphates, pyrophosphoric acid, fishbone phosphate, animal bone phosphate, herring meal, bone meal, phosphorites, and combinations thereof. Salts of phosphoric acid can be used and are preferably alkali metal salts such as, but not limited to, trisodium phosphate, dicalcium phosphate, disodium hydrogen phosphate, sodium dihydrogen phosphate, tripotassium phosphate, dipotassium hydrogen phosphate, potassium dihydrogen phosphate, trilithium phosphate, dilithium hydrogen phosphate, lithium dihydrogen phosphate or mixtures thereof.

The amounts and type of stabilizing agent and combinations, according to the method of invention, depend on various factors including desired solubility reduction potential, desired mineral toxicity, and desired mineral formation relating to stomach acid control objectives. It has been found that an amount of certain stabilizing agents such as fifty (50) percent tricalcium phosphate by weight of total soluble lead introduced into a HCL stomach simulation CFSTR is sufficient for soluble Pb stabilization to less than 5.0 ppb. However, the foregoing is not intended to preclude yet higher or lower usage of stabilizing agent and combinations. The examples below are merely illustrative of this invention and are not intended to limit it thereby in any way.

### EXAMPLE 1

In this example, a 1000 ppm soluble lead chloride solution was produced by addition of lead chloride to 1000 ml of 1.0 Normal HCL solution. Various stabilizers were added to the HCL extractor environment in solid tablet form. Tablets were produced by combination of equal parts by weight of 20% sugar water and dry stabilizer, hand mixing for 10 seconds, and subsequent hand press tablet forming. Stabilizers included Tricalcium Phosphate (TCP), Dicalcium Phosphate (DCP), Potassium Chloride (KCI), Sodium Chloride (NaCl2) and Iron Oxide Powder (FeO).

Each separate sample environment was extracted according to TCLP procedure set forth in Federal Register, Vol. 55, No. 126, pp. 26985-26998 (Jun. 29, 199), modified by use of 1.0 N HCL acid replacing acetic acid solution, and adjusting the extraction period to 48 hours versus the method 18 hours, which is hereby incorporated by reference. A modified 48 hour extract leachate was digested prior to analysis by ICP.

TABLE 1

| | | Reactor Pb |
|---|---|---|
| Stabilizer Recipe | Stabilizer Dosage (gram) | Content (ppm) |
| TCP | 0.5 | 3.00 |
| TCP | 1 | <0.05 |
| DCP | 0.5 | 5.01 |
| DCP | 1 | <0.05 |
| DCP + NaCl2 | 0.5 | <0.05 |
| DCP + NaCl2 | 1 | <0.05 |
| FeO + KCl + DCP | 1 | <0.05 |

The foregoing results in Table 1 readily established the operability of the present process to reduce soluble lead content in an HCL leaching environment simulating the human stomach. Given the effectiveness of the stabilizing agent as presented in Table 1, it is believed that an amount of the stabilizing agent and combinations to less than 50% by weight of the heavy metal in solution should be effective.

While this invention has been particularly shown and described with reference to preferred embodiments thereof, it will be understood by those skilled in the art that various changes in form and details may be made therein without departing from the spirit and scope of the invention as defined by the appended claims.

I claim:

1. A method of reducing the solubility of heavy metal within the human stomach, comprising oral ingestion of at least one stabilizing agent in an amount effective in reducing the solubility of heavy metal to desired levels.

2. The method of claim 1, wherein the stabilizing agent is selected from the group consisting of phosphates, phosphoric acid, technical grade phosphoric acid, wet process produced amber or green phosphoric acid, potassium chloride, sodium chloride, calcium chloride, iron powder, ferric sulfate, ferrous sulfate, ferric chloride, hexametaphosphate, polyphosphate, calcium orthophosphate, superphosphates, triple superphosphates, phosphate rock, bone phosphate, fishbone phosphates, tetrapotassium polyphosphate, monocalcium phosphate, monoammonia phosphate, diammonium phosphate, dicalcium phosphate, tricalcium phosphate, trisodium phosphate, salts of phosphoric acid, and combinations thereof.
